(19) United States
(12) Patent Application Publication
(10) Pub. No.: US 2013/0005598 A1
(43) Pub. Date: Jan. 3, 2013

(54) Methods for Diagnosing The Malignant Potential of Pancreatic Cystic Lesions

(75) Inventors: Brian B. Haab, Jenison, MI (US); Diane Simeone, Ann Arbor, MI (US)

(73) Assignees: The Regents of the University of Michigan, Ann Arbor, MI (US); Van Andel Research Institute, Grand Rapids, MI (US)

(21) Appl. No.: 13/519,766

(22) PCT Filed: Dec. 30, 2010
(86) PCT No.: PCT/US10/62534

§ 371 Date: Sep. 10, 2012

Related U.S. Application Data
(60) Provisional application No. 61/291,654, filed on Dec. 31, 2009.

(51) Int. Cl. G01N 33/574; C40B 30/04; A61B 5/153; C40B 40/10

(52) U.S. Cl. 506/9; 506/18; 435/7.9; 600/578

(57) ABSTRACT

A method of diagnosing the malignant potential of a pancreatic cystic lesion in a subject including: detecting a glycan alteration in MUC5AC in a sample of pancreatic cystic lesion fluid from a subject, determining whether the glycan alteration is differentially present in the sample, and diagnosing the malignant potential of the pancreatic cystic lesion. A method of diagnosing the malignant potential of a pancreatic cystic lesion in a subject including: (a) detecting a glycan alteration in MUC5AC in a sample of pancreatic cystic lesion fluid from a subject, (b) detecting CA 19-9 in the sample, (c) determining whether the glycan alteration and CA 19-9 are differentially present in the sample, and (d) diagnosing the malignant potential of the pancreatic cystic lesion. Related methods of treatment and kits also are included.

### CROSS-REFERENCE TO RELATED APPLICATION

The present application claims the benefit of provisional application Ser. No. 61/291,654 filed Dec. 31, 2009, entitled METHODS FOR DIAGNOSING THE MALIGNANT POTENTIAL OF PANCREATIC CYSTIC LESIONS, the entire contents of which is incorporated herein in its entirety.

### STATEMENT REGARDING FEDERALLY SPONSORED RESEARCH OR DEVELOPMENT

This invention was in part made with United States government support awarded by National Institute of Health (Grant Numbers NC1 R21 CA122890 and R33 CA122890). The U.S. Government has a paid-up license in the invention and the right in limited circumstances to require the patent owner to license others on reasonable terms.

### FIELD OF THE INVENTION

This invention relates to the field of molecular biology and medicine and specifically to diagnosing pancreatic cancer.

### BACKGROUND OF THE INVENTION

Cystic lesions of the pancreas are increasingly being recognized due to the widespread use of high resolution abdominal imaging. Since certain cyst types are precursors to invasive cancer, this situation presents an opportunity to intervene prior to malignant progression. Effective implementation of that strategy has been hampered by difficulties in clearly distinguishing cystic lesions with no malignant potential from those with malignant potential.

The development of effective diagnostic and treatment strategies for pancreatic cancer has been extremely challenging. Because of the difficulty in detecting pancreatic cancer at early stages, most cancers are advanced at the time of diagnosis and refractory to existing treatment. The detection and surgical removal of locally invasive cancer results in improved survival rates, but the cancer still recurs in most patients. The cause of recurrence is most likely due to the early escape from the primary tumor, prior to surgery, of metastatic cancer cells that eventually develop into advanced disease. Since micrometastatic cancer can occur at such early stages of the primary tumor, the best hope for long-term cures of pancreatic cancer may be the surgical removal of pre-malignant precursor lesions that have not yet developed into invasive cancer²⁻⁴. However, effective means to routinely detect pre-invasive pancreatic neoplasms do not currently exist.

Recent research has provided firm evidence for the stepwise development of pancreatic ductal adenocarcinomas—the most common and deadly form of pancreatic cancer—from three types of precursor lesions². The most prevalent precursor type is pancreatic intraepithelial neoplasia (PanIN)^{5,6}, which arises in the epithelial cells of pancreatic ducts. It is not yet possible to detect PanINs for screening purposes since they are too small to be seen by imaging and are not associated with any secreted biomarker. The other two precursor lesions are mucinous cystic neoplasms (MCN) and intraductal papillary mucinous neoplasms (IPMN). While these precursors are rarer than PanINs, they account for the development of up to 10-15% of pancreatic cancers⁷. Unlike PanINs, they can be detected by CT or ultrasound imaging, giving the possibility of detecting and removing these cancer precursors prior to the development of invasive cancer⁴. With the current widespread use of high resolution abdominal imaging, pancreatic cystic tumors are increasing being identified, many of which are in the asymptomatic patient⁸. As many as 1% of abdominal CT scans reveal pancreatic cysts⁹, with that number potentially increasing as the resolution of imaging technology improves. This detection of pancreatic “incidentalomas” presents an opportunity to reduce pancreatic cancer mortality through the removal of these precursor lesions prior to the development of invasive cancer. However, certain diagnostic challenges need to be addressed before that strategy could make a significant impact on pancreatic cancer.

A major challenge in diagnosing pancreatic cystic lesions arises from the fact that certain benign cyst types which have no potential to progress to cancer (i.e., no malignant potential) are sometimes difficult to distinguish from the MCN and IPMN cancer precursors. It is important to accurately make this distinction so that surgical removal is performed only in patients in whom resection is beneficial. The two most common types of such benign cystic lesions found in the pancreas are pancreatic pseudocysts (PC) and serous cystadenomas (SC). Unfortunately, current methods of evaluating cystic pancreatic lesions are limited in their ability to differentiate pseudocysts from mucin-containing cystic tumors. In addition, preoperative imaging or endoscopic studies do not reliably differentiate between serous and mucin-producing neoplasms. Although the use of endoscopic ultrasound (EUS) and analysis of cyst fluid can generally differentiate between the two types, accuracy of no greater than 79% is reported in the literature¹⁰.

A promising means of diagnosing the type of cystic tumor is the analysis of the fluid trapped inside the cyst, which can be collected by endoscopic ultrasound fine-needle aspiration (EUS-FNA). The cytologic examination of cyst fluid has low diagnostic sensitivity¹¹, presumably because of the paucity of tumor cells within the cyst itself. Recently, molecular studies have been performed on cyst fluid samples in order to discover biomarkers secreted by the encapsulating epithelial cells that are indicative of the type of cyst. Thus far, the most accurate biomarker is carcinoembryonic antigen (CEA). A combined analysis of 12 different studies found that CEA distinguished mucin-producing (not including IPMN) from benign cysts with an average 48% sensitivity and 98% specificity¹¹. Other types of biomarkers that have been tested in cyst fluid include DNA quality and mutations¹², tumor-associated trypsin inhibitor¹³, and the presence of mucin^{14,15}. Despite great initial enthusiasm for the commercially available REDPATH™ evaluation of cyst fluid, this DNA analysis appears to have significant limitations to accurately select patients who require surgery, and has not replaced CEA testing for routine diagnostic analysis¹⁶. As set forth below, the inventors now have discovered glycosylation variants on specific proteins in cyst fluid samples that could serve as biomarkers to aid in this diagnosis.

### SUMMARY OF THE INVENTION

The inventors have found that certain mucin and CEA-family proteins and their glycan variants have potential as biomarkers for the accurate diagnosis of pancreatic cystic lesions. In particular, the inventors utilized a novel antibody-lectin sandwich microarray method to measure the protein expression and glycosylation of MUC1, MUC5AC, MUC16, CEA, and other proteins implicated in pancreatic neoplasia in cyst fluid samples. The detection of a glycan variant on MUC5AC using the lectin wheat-germ agglutinin discriminated mucin-producing cystic tumors (mucinous cystic neoplasms and intraductal papillary mucinous neoplasms) from benign cystic lesions (serous cystadenomas and pseudocysts) with a 78% sensitivity at 80% specificity, and when used in combination with cyst fluid CA 19-9 gave a sensitivity of 87% at 86% specificity, significantly better than the performance of CEA. This finding will allow for more accurate diagnosis of pancreatic cystic lesions in patients who will benefit from surgical intervention.

The present invention includes a method of diagnosing the malignant potential of a pancreatic cystic lesion in a subject including: (a) obtaining a pancreatic cyst fluid sample from a pancreatic cystic lesion in a subject, (b) detecting a glycan alteration in MUC5AC in the sample, (c) determining whether the glycan alteration is differentially present in the sample, and (d) diagnosing the malignant potential of the cystic lesion. In one embodiment, if it is determined that the glycan alteration is present at a higher level in the sample as compared to pancreatic cystic lesions having no malignant potential, then the cystic lesion is diagnosed as having malignant potential. Further, the glycan alteration may detected by a lectin, such as Vicia villosa, Jacalin, wheat-germ agglutinin (WGA), and Erythrina cristagalli lectin (ECL). In another embodiment, the sample may be obtained by endoscopic ultrasound fine-needle aspiration.

The present invention also includes a method of diagnosing the malignant potential of a pancreatic cystic lesion in a subject which includes: (a) obtaining a pancreatic cyst fluid sample from a pancreatic cystic lesion in a subject, (b) detecting a glycan alteration in MUC5AC in the sample, (c) detecting CA 19-9 in the sample, (d) determining whether the glycan alteration and CA 19-9 are differentially present in the sample, and (e) diagnosing the malignant potential of the cystic lesion. In one embodiment, if it is determined that the glycan alteration and CA 19-9 are present at higher levels in the sample as compared to pancreatic cystic lesions having no malignant potential, then the cystic lesion is diagnosed as having malignant potential.

The present invention also includes a method for determining the malignant potential of a pancreatic cyst lesion, including: obtaining a pancreatic cyst fluid sample from a patient having or suspected of having a pancreatic disease; assaying the sample for a glycan level of MUC5AC in the sample; comparing the glycan level in MUC5AC in the sample to a statistically validated threshold for MUC5AC, which statistically validated threshold for MUC5AC is based on a glycan level in MUC5AC in comparable control samples from benign pancreatic cysts in subjects, wherein a different glycan level in MUC5AC in the sample as compared to the statistically validated threshold indicates that the pancreatic cyst lesion from which the sample was obtained has malignant potential. This method also may include: assaying the sample for a CA 19-9 level in the sample; and comparing the level of CA 19-9 antigen in the sample to a statistically validated threshold for CA 19-9 antigen, which statistically validated threshold for CA 19-9 antigen is based on a level of CA 19-9 antigen in comparable control samples from benign pancreatic cysts in subjects; wherein (a) a different level of CA 19-9 antigen in the sample as compared to the statistically validated threshold for CA 19-9 antigen and (b) a different level of glycan level in the MUC5AC in the sample as compared to the statistically validated threshold for the MUC5AC indicate that the pancreatic cyst lesion from which the sample was obtained has malignant potential.

Further, the present invention includes a method for treating a pancreatic cystic lesion in a patient comprising: obtaining a pancreatic cyst fluid sample from a pancreatic cystic lesion in a patient, assaying the sample for a glycan level of MUC5AC; determining whether the glycan level of MUC5AC in the sample is present at a higher level than the glycan level of MUC5AC in pancreatic cystic lesions having no malignant potential; and surgically removing the pancreatic cystic lesion from the patient if the glycan level of MUC5AC in the sample is present at the higher level. With this method, The method of claim 10, the glycan level of MUC5AC may be assayed with a lectin, the lectin may be Vicia villosa, Jacalin, wheat-germ agglutinin (WGA), and Erythrina cristagalli lectin (ECL), and the sample may be obtained by endoscopic ultrasound fine-needle aspiration. Further, this method also may include: assaying the sample for a level of CA 19-9; determining whether the level of CA 19-9 in the sample is present at a higher level than the level of CA 19-9 in pancreatic cystic lesions having no malignant potential; and surgically removing the pancreatic cystic lesion from the patient if the glycan level of MUC5AC in the sample is present at a higher level than the glycan level of MUC5AC in pancreatic cystic lesions having no malignant potential and the level of CA 19-9 in the sample is present at a higher level than the level of CA 19-9 in pancreatic cystic lesions having no malignant potential.

Another aspect of the present invention is a method for treating a pancreatic cystic lesion in a patient comprising: obtaining a pancreatic cyst fluid sample from a patient having or suspected of having a pancreatic disease; assaying the sample for a glycan level of MUC5AC in the sample; comparing the glycan level in MUC5AC in the sample to a statistically validated threshold for MUC5AC, which statistically validated threshold for MUC5AC is based on a glycan level in MUC5AC in comparable control samples from benign pancreatic cysts in subjects; and surgically removing the pancreatic cystic lesion from the patient if the glycan level in MUC5AC in the sample is different than the statistically validated threshold. With this method, the glycan level of MUC5AC may be assayed with a lectin, the lectin may be Vicia villosa, Jacalin, wheat-germ agglutinin (WGA), and Erythrina cristagalli lectin (ECL), and the sample may be obtained by endoscopic ultrasound fine-needle aspiration. Additionally, this method may include: assaying the sample for a level of CA 19-9; comparing the glycan level of CA 19-9 in the sample to a statistically validated threshold for CA 19-9, which statistically validated threshold for CA 19-9 is based on a level of CA 19-9 in comparable control samples from benign pancreatic cysts in subjects; and surgically removing the pancreatic cystic lesion from the patient if the glycan level in MUC5AC in the sample is different than the statistically validated threshold for MUC5AC, and if the level of CA 19-9 in the sample is different than the statistically validated threshold for CA 19-9.

The present invention also includes a kit, which kit has: (a) an antibody microarray with an anti-CA 19-9 capture antibody and an anti-MUC5AC capture antibody bound thereto, (b) a detection antibody to the CA-19-9 antigen, (c) a detection antibody to a MUC5AC glycan, and (d) one or more containers for such detection antibodies.

### BRIEF DESCRIPTION OF THE DRAWING

The foregoing summary, as well as the following detailed description of the invention, will be better understood when read in conjunction with the appended drawings. For the purpose of illustrating the invention, there are shown in the drawings, certain embodiment(s) which are presently preferred. It should be understood, however, that the invention is not limited to the precise arrangements and instrumentalities shown.

FIGS. 1A-1D show protein and glycan detection on antibody arrays. FIG. 1A is a drawing showing array-based sandwich assays for protein detection. Multiple antibodies are immobilized on a planar support, and the captured proteins are probed using biotinylated detection antibodies, followed by fluorescence detection using phycoerythrin-labeled streptavidin. FIG. 1B is a drawing showing glycan detection on antibody arrays. This format is similar to FIG. 1A, but the detection reagents target the glycans on the capture proteins rather than the core proteins. The glycans on the immobilized antibodies are chemically derivatized to prevent lectin binding to those glycans. FIG. 1C is a drawing showing high-throughput sample processing. Forty-eight or sixty identical microarrays are printed on one microscope slide, segregated by hydrophobic boundaries. A set of serum samples is incubated on the arrays in a random order, and each slide is probed with a single antibody or lectin. FIG. 1D shows exemplary antibody array results for specific capture antibodies (indicated at left) and detection reagents (indicated in the column labels), after incubation with the indicated samples.

FIG. 2 shows a cluster analysis of antibody-lectin sandwich array results. Measurements showing significant differences (p<0.02) between mucin-producing cystic neoplasms (MCN and IPMN) to non-mucinous cysts (SC and PC) are presented. Each square represents the signal level from a sample (indicated by the column labels) detected with a particular capture antibody and detection reagent (indicated by the row labels). Each column label gives the diagnosis and a patient identifier, and the color of the label indicates whether the sample is a mucin-producing cystic neoplasms (MCN or IPMN, red) or non-mucinous cyst (SC or PC, green). The fluorescence values were log-transformed (base 10) and median-centered along each row in order to clearly show the variation between the samples. The color bar gives the scale, in which each unit represents a 10-fold change.

FIGS. 3A-3D are box plots indicating the levels of particular markers in each class. Each point represents an individual sample. The boxes indicate the quartiles, with the median indicated by the horizontal lines, and the vertical lines give the ranges. FIG. 3A shows WGA detection at the MUC5AC capture antibody. FIG. 3B shows CEA levels; FIG. 3C shows CA 19-9 levels; and FIG. 3D shows MUC1 levels.

FIGS. 4A and 4B show comparisons of results from antibody microarrays to Western blots. FIG. 4A show comparisons of MUC5AC levels. The antibody microarray results are represented by the column graphs for the indicated capture and detection antibodies and the indicated samples. The corresponding samples were separated by SDS-PAGE (with the lane order matching the column graphs), blotted, and probed using the indicated detection antibodies. Lysates from cell lines were analyzed in the right lanes. The region of the separations containing the molecular weights expected for MUC5AC are indicated by the blue boxes. FIG. 4B shows comparisons of CEA levels.

FIGS. 5A and 5B show scatter plots discriminating patient groups using individual and combined markers. FIG. 5A is a scatter plot comparison of two biomarkers. Each point represents a sample, with the color of each point indicating its class, according to the legend. The y-axis represents the level of CA19-9, and the x-axis represents the level of WGA-MUC5AC. The dashed lines are the thresholds used to dichotomize the samples for each marker. FIG. 5B shows receiver-operator characteristic (ROC) curves for the discrimination of mucin-producing cystic tumors (MCN and IPMN) from non-mucinous (SC and PC) cysts. The area-under-the-curve (AUC) and 95% confidence interval are indicated for CEA, WGA-MUC5AC, and the combination of WGA-MUC5AC and CA 19-9. For the combined biomarker, each sample was classified as mucin-producing if the level of either biomarker was above its threshold indicated by the dashed lines in FIG. 5A.

### DETAILED DESCRIPTION OF THE PREFERRED EMBODIMENTS

The details of one or more embodiments of the invention are set forth in the accompanying drawings and the description below. Other features, embodiments, and advantages of the invention will be apparent from the description and drawings, and from the claims. The preferred embodiments of the present invention may be understood more readily by reference to the following detailed description of the specific embodiments and the Examples included hereafter. It is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

All references, patents, patent publications, articles, and databases, referred to in this application are incorporated herein by reference in their entirety, as if each were specifically and individually incorporated herein by reference. Such patents, patent publications, articles, and databases are incorporated for the purpose of describing and disclosing the subject components of the invention that are described in those patents, patent publications, articles, and databases, which components might be used in connection with the presently described invention. The information provided below is not admitted to be prior art to the present invention, but is provided solely to assist the understanding of the reader.

For clarity of disclosure, and not by way of limitation, the detailed description of the invention is divided into the subsections that follow.

Unless defined otherwise, all technical and scientific terms used herein have the meaning commonly understood by one of ordinary skill in the art to which this invention belongs. Generally, the nomenclature used herein and the laboratory procedures in cell culture, molecular genetics, organic chemistry and nucleic acid chemistry described below are those well known and commonly employed in the art. Although any methods, devices and materials similar or equivalent to those described herein can be used in the practice or testing of the invention, the preferred methods, devices and materials are now described.

Definitions

In this specification and the appended claims, the singular forms “a,” “an” and “the” include plural reference unless the context clearly dictates otherwise.

“Detect” refers to identifying the presence, absence or amount of the object to be detected.

The phrase “differentially present” refers to a difference in the quantity and/or the frequency of a protein(s), a polypeptide(s), a glycan alteration(s), or a carbohydrate epitope(s) present in samples taken from pancreatic cystic lesions having malignant potential as compared to samples taken from pancreatic cystic lesions having no malignant potential. For example, a protein(s), a polypeptide(s), a glycan alteration(s), or a carbohydrate epitope(s) may be differentially present in that it is present at an elevated level in samples from pancreatic cystic lesions having malignant potential as compared to samples from pancreatic cystic lesions having no malignant potential. A protein(s), a polypeptide(s), a glycan alteration(s), or a carbohydrate epitope(s) can be differentially present in terms of quantity, frequency or both. For the purpose of this invention, a protein(s), a polypeptide(s), a glycan alteration(s), or a carbohydrate epitope(s) is differentially present when there is at least an about a two-fold, preferably at least about a four-fold, more preferably at least about a six-fold, most preferably at least about a ten-fold difference between the quantity and/or frequency of a given protein(s), polypeptide(s), glycan alteration(s), or carbohydrate epitope(s) in pancreatic cystic lesions having malignant potential as compared to pancreatic cyst lesions have no malignant potential.

A “glycan alteration” means a change in glycosylation state in a protein or polypeptide including, but not limited to, an addition, deletion, substitution, truncation, branching, or chain extension of a carbohydrate group.

A tissue has “malignant potential” if that tissue is likely to progress to cancer or already is cancerous. For example, a pancreatic cyst has malignant potential if that cyst is likely to develop into a mucinous cystic neoplasm or an intraductal papillary mucinous neoplasm.

“Pancreatic cyst fluid sample” means any fluid derived from a cystic lesion of the pancreas of a subject.

As used herein, a “subject” or “patient” is a warm blooded mammal, including, humans, farm animals such as horses, sheep, cattle, lamas, pigs and the like, as well as pets such as cats and dogs. In one embodiment, the warm blooded mammal is a human.

The term “treatment” or “treating” as used herein refers to the administration of medicine or the performance of a medical procedure with respect to a patient, for either prophylaxis (prevention) or to cure or reduce the extent of or likelihood of occurrence or recurrence of the infirmity or malady or condition or event in the instance where the patient is afflicted. As related to the present invention, the term may also mean the administration of medicine or the performance of a medical procedure as therapy, prevention or prophylaxis of pancreatic cancer, e.g., the surgical removal of a pre-malignant precursor lesion.

One novel strategy for the development of biomarkers for pancreatic cancer is to analyze carbohydrate alterations associated with particular proteins found in the cyst fluid. Changes to glycans on proteins are common in pancreatic cancer and are thought to play functional roles in the disease¹⁷⁻²⁰. The detection of carbohydrate changes may yield more effective biomarkers relative to measurements of core protein levels because they may be altered more reliably. The value of measuring glycan variants for biomarker discovery in several cancer types has been demonstrated in studies, including elevated fucose levels on haptoglobin²¹⁻²³ in breast, ovarian cancer and pancreatic cancer, on alpha-1-antitrypsin²⁴ in ovarian cancer, and on alpha-fetoprotein^{25,26} in hepatocellular carcinoma. In addition, glycan-based biomarkers in the serum of pancreatic cancer patients have shown considerable promise^{27,28}.

A particularly valuable platform for probing glycan variants on multiple, specific proteins in biological samples is the antibody-lectin sandwich microarray^{29,30}. This approach complements previous technologies by enabling the sensitive probing of changes to particular glycan structures on specific proteins, which is not possible using mass spectrometry or chromatographic methods. Unlike conventional methods, microarray methods require only small amounts of sample (typically 6 μl of fluid after dilution), making them suitable for studies on cyst fluid. Furthermore, antibody microarrays can be run in a high-throughput fashion, so that population-based studies can be performed to assess biomarker potential.

Here, the inventors discovered differences in the abundance or glycosylation of particular proteins in cyst fluid between mucin-producing cystic tumors (MCNs and IPMNs) and benign cystic lesions. The inventors designed antibody microarrays to target proteins that are known to be secreted by cancer cells and that often display altered glycosylation¹⁸, including the mucins MUC1, MUC5AC, and MUC16, and CEA. The antibody microarrays were processed to measure either the abundance or the glycosylation state of these proteins in cyst fluid samples from patients with MCNs, IPMNs, SCs or PCs. The inventors demonstrated that specific molecular features which characterize the fluid of each of these states can form the basis of biomarkers that improve the accuracy of differentiating pancreatic cystic tumor type.

The increased incidental detection of pancreatic cystic tumors presents a significant opportunity to reduce mortality from pancreatic cancer, since potentially life-threatening neoplasms could be removed prior to reaching an invasive stage. The key to capitalizing on that opportunity is the accurate differentiation of cystic lesions, so that the patients most likely to benefit from operative intervention can be optimally identified. The present work revealed significant differences between mucin-producing cystic tumors and non-mucinous cysts in the levels and glycan variants of MUC5AC, CEACAM6, MUC1, fibronectin, CEA, and CA 19-9. Certain markers performed better than CEA, and because of complementary patterns in some of the biomarkers, additional accuracy was achieved by using them in combination.

This work made use of a novel antibody-lectin sandwich array technology that allowed convenient analysis of protein abundance and glycosylation in multiple biosamples. Affinity-based approaches such as this have the advantages of sensitively detecting analytes directly out of small volumes of complex mixtures with high reproducibility and high sample throughput³⁵. The combination of antibody-based capture with lectin detection of glycan levels may be especially useful for pancreatic cancer biomarker discovery, since glycosylation alterations are a common feature of pancreatic cancers, and because glycan changes may occur more frequently or to a higher degree than changes in protein concentrations. This relationship was suggested in previous studies on serum proteins in pancreatic cancer by the inventors and others²⁷⁻²⁹, and seems to be the case here, since lectin detection of MUC5AC and CEACAM6 provided better performance than antibody detection of the core proteins (Table 3).

The fact that certain biomarkers were highly complementary resulted in improved performance when used in combination relative to their individual use. CA 19-9 was predominantly elevated in MCNs, while WGA-MUC5AC identified a higher proportion of IPMNs, resulting in an 87%/86% sensitivity/specificity when used together. Such performance may meet the requirements for identifying most patients at high risk for developing cancer while not admitting a high rate of false-positive identification.

The inventors observed that CEA was not elevated in serous cystadenomas but was elevated in a high proportion of pseudocysts. This distinction in CEA levels between these two types of benign cysts may result in improving the usefulness of CEA if the diagnosis of pseudocyst could be clearly discounted, which may be possible in some patients. Although previous studies did not specifically address the differential levels of CEA in PC and SC, it was suggested by a combined analysis of 12 studies that showed a higher median value in PC (10 ng/mL, n=125) than in SC (3 ng/mL, n=79)¹¹. If pseudocysts were excluded in the inventors' analysis, the combination of CEA, which was elevated in most of the mucin-producing cystic tumors, and ECL-CEACAM6, which was elevated in the non-mucinous cysts, gave a sensitivity of 100% at 80% specificity. These biomarkers may be valuable when clinical or other information discounts the diagnosis of pseudocyst.

A glycan-binding protein or peptide ligand, such as a lectin, may be used to detect glycosylation levels of a target protein; and the binding specificities of the lectins utilized in this study can provide insight into the nature of the altered glycans. For example, the lectin Vicia villosa (VVL) has specificity for terminal galactosamine (GalNAc), and the increased binding of VVL on MUCSAC from mucin-producing cystic tumors may be due to truncation of O-glycans at the core GalNAc. GalNAc attached to the serine or threonine residue, referred to as the Tn antigen, has been frequently associated with pancreatic cancer and other cancers³⁶⁻³⁸. The Jacalin lectin, which also showed high binding to MUCSAC from mucin-producing cystic tumors, can bind the Tn antigen as well as the related T antigen (Galb1,3GalNAc), which also is strongly associated with cancer³⁹. The lectin WGA binds N-acetlyglucosamine (GlcNAc) and other saccharides. Increased GlcNAc could be due to increased branching of O-glycans or N-glycans, resulting in increased extension of glycan chains through repeated lactosamine (Galβ1,4GlcNAcβ1,3) units. Both N-glycan branching^{40, 41} and O-glycan branching⁴² are associated with the formation of cancer-associated glycans such as the Lewis blood group structures⁴². The Lewis blood group structures are ligands for selectin receptors found on endothelial cells and lymphocytes⁴³, and increased presentation of this structure on pancreatic cells leads to increased metastasis ⁴⁴⁻⁴⁶ and reduced survival in pancreatic cancer^{47,48}. The Erythrina cristagalli lectin (ECL), which showed high binding to both MUC5AC and CEACAM6 from mucinous cysts, also binds lactosamine, which is consistent with the results using WGA. Therefore, it appears that the inventors observed both the truncation of certain O-glycans as well as increased chain extension on others, perhaps on N-glycans.

Various detectable labels, such as biotin, can be utilized to detect the glycan-binding protein that is bound to the glycan. Generally, suitable detectable labels include radioactive, fluorescent, fluorogenic, chromogenic, or other chemical labels. Useful radio labels, which are detected by gamma counter, scintillation counter, or auto radiography include ³H, ¹²⁵I, ¹³¹I, ³⁵S, and ¹⁴C. Common fluorescent labels include fluorescein, rhodamine, dansyl, phycoerythrin, phycocyanin, allophycocyanin, o phthaldehyde, and fluoroescamine. The fluorophoor, such as the dansyl group, must be excited by light of a particular wavelength to fluoresce. The protein can also be labeled for detection using fluorescence-emitting metals such as ¹⁵²Eu, or others of the lanthanide series.

In the practice of the present invention, diagnostic and prognostic methods may be based upon the steps of obtaining a pancreatic cyst fluid sample from a pancreatic cyst in a subject, contacting the sample with a glycan-binding protein, such as a lectin (to detect a glycan alteration in MUC5AC) and/or an antibody, or fragment thereof (to detect a CA 19-9), detecting a glycan alteration in MUC5AC in the sample and/or detecting CA 19-9 in the sample, determining whether the glycan alteration and/or CA 19-9 are differentially present in the sample, and diagnosing the malignant potential of the cystic lesion.

The CA 19-9 marker is a carbohydrate antigen that is detected by a monoclonal antibody. This carbohydrate antigen, a quatra-saccharide called sialy Lewis A, is found on multiple, different proteins. In the sandwich ELISA method used in a clinical assay, the CA 19-9 monoclonal antibody measures the CA 19-9 antigen on many different carrier proteins.

A glycan alteration can be detected by various known methods. One example is the detection of a glycan alteration by a glycan-binding protein, such as a lectin. Lectins include carbohydrate-binding proteins from many sources regardless of their ability to agglutinate cells. Lectins have been found in many organisms, including, plants, viruses, microorganisms and animals. Most known lectins are multimeric, with non-covalently associated subunits, and this multimeric structure gives lectins their ability to agglutinate cells or form precipitates with glycoconjugates similar to antigen-antibody interactions. A common characteristic of lectins is that they bind to specifically defined carbohydrate structures. Because of this specificity that each lectin has for a particular carbohydrate structure, even oligosaccharides with identical sugar compositions can be distinguished. Some lectins bind only structures with mannose or glucose residues, while others recognize only galactose residues. Some lectins bind only if a particular sugar is in a terminal non-reducing position in the oligosaccharide, while others bind sugars within the oligosaccharide chain. Further, some lectins do not discriminate when binding to a and b anomers, while other lectins require the correct anomeric structure and a specific sequence of sugars. Thus, the binding affinity between a lectin and its receptor may vary greatly in view of seemingly small changes in the carbohydrate structure of the receptor.

In certain aspects, the invention provides assays and methods to discriminate cystic pancreatic tumors from benign cystic lesions. That is, the present methods and assays may be used to diagnose, prognose, and treat a cancerous pancreatic lesion. In some embodiments, these methods and assays detect glycan alterations on MUC5AC in a pancreatic cyst fluid sample and, in other embodiments, glycan alterations on MUC5AC are detected as a complementary biomarker to CA 19-9 levels in the sample. Any assay that will detect total CA 19-9 and MUC5AC glycan levels can be used, whether assayed individually (e.g., by sandwich ELISA or other methods known in the art) or by high throughput methods (e.g., by using antibody arrays such as those described herein).

Further, the present invention provides a method for diagnosing the malignant potential of a pancreatic cystic lesion in a subject, said method comprising screening for levels of glycan alteration in MUC5AC and/or of CA 19-9 antigen in pancreatic cyst fluid, wherein a difference in the level of the glycan alteration in MUC5AC and/or of CA 19-9 antigen compared to a statistically validated threshold is indicative of the malignant potential of the pancreatic cystic lesion in the subject. The statistically validated threshold may be based upon levels of biomarkers, in comparable samples obtained from a control population, e.g., the general population, or a select population of human subjects, such as subjects having pancreatic pseudocysts and serous cystadenomas. For example, the select population may be comprised of apparently healthy subjects. “Apparently healthy”, as used herein, means individuals who have not previously had any signs or symptoms indicating the presence of malignant pancreatic cancer, including mucinous cystic neoplasms (MCN) and intraductal papillary mucinous neoplasms (IPMN).

The statistically validated threshold is related to the value used to characterize the level of the biomarker obtained from the subject. Thus, if the level of the biomarker is an absolute value, then the control value is also based upon an absolute value.

The statistically validated threshold can take a variety of forms. The statistically validated threshold can be a single cut-off value, such as a median or mean. The statistically validated threshold can be established based upon comparative groups such as where the risk in one defined group is double the risk in another defined group. The statistically validated threshold can be divided equally (or unequally) into groups, such as a low risk group, a medium risk group and a high-risk group, or into quadrants, the lowest quadrant being individuals with the lowest risk the highest quadrant being individuals with the highest risk, and the subject's risk of having pancreatic cancer or a predisposition to develop pancreatic cancer can be based upon which group his or her test value falls.

Statistically validated threshold of the biomarkers obtained, such as for example, mean levels, median levels, or “cut-off” levels, may be established by assaying a large sample of individuals in the general population or the select population and using a statistical model such as the predictive value method for selecting a positivity criterion or receiver operator characteristic curve that defines optimum specificity (highest true negative rate) and sensitivity (highest true positive rate) as described in Knapp, R. G., and Miller, M. C. (1992). Clinical Epidemiology and Biostatistics. William and Wilkins, Harual Publishing Co. Malvern, Pa., which is specifically incorporated herein by reference. A “cutoff value” can be determined for each biomarker that is assayed.

Levels of each select biomarker in the pancreatic cyst fluid sample may be compared to a single control value or to a range of control values. If the level of the biomarker in the sample is different than the statistically validated threshold, the test subject is at greater risk of developing or having pancreatic cancer than individuals with levels comparable to the statistically validated threshold. The extent of the difference between the subject's biomarker(s) levels and statistically validated threshold is also useful for characterizing the extent of the risk and thereby, determining which individuals would most greatly benefit from certain aggressive therapies. In those cases, where the statistically validated threshold ranges are divided into a plurality of groups, such as the statistically validated threshold ranges for individuals at high risk, average risk and low risk, the comparison involves determining into which group the subject's level of the relevant risk predictor falls.

Another embodiment of the present invention is a diagnostic kit for discriminating cystic pancreatic tumors from benign cystic lesions. In one embodiment, a biomarker panel or array (as described herein) is provided to distinguish a cystic pancreatic tumor from a benign cystic lesion. The inventive kit for differentiating cystic pancreatic tumors from benign cystic lesions may include (a) an antibody array having an anti-CA 19-9 capture antibody bound thereto and/or an anti-MUC5AC capture antibody bound thereto, (b) a detection antibody to the CA-19-9 antigen and/or a detection antibody or glycan-binding protein to MUC5AC to detect levels of CA 19-9 and/or MUC5AC glycan levels in a sample, respectively; and (c) one or more containers for such detection reagents. For example, the diagnostic kit could include WGA to detect a glycan variant on MUC5AC (alone or in combination with an antibody, or antibody fragment, that is specific to CA 19-9 to detect CA 19-9) and instructions to use the kit, as an early stage screen to differentiate benign pancreatic cysts from pancreatic cysts that have the potential to progress to pancreatic cancer.

Diagnostic kits of the present invention can include any appropriate glycan binding protein. Some examples include Aleuria Aurantia lectin (AAL), Wheat Germ Agglutinin (WGA), Jacalin, Bauhinea Purpurea lectin (BPL), Sambucus Nigra lectin (SNA), a glycan-binding antibody, or other glycan binding antibodies described herein or otherwise known in the art.

In another embodiment, the inventive diagnostic kit includes capture and detection antibodies (or other glycan binding proteins) to capture and detect CA 19-9 and/or MUC5AC glycan levels using sandwich ELISA, or other methods known in the art, to individually detect MUC5AC glycan levels and/or CA 19-9 levels. The inventive kits may be used to perform the methods described herein.

The present predictive tests are useful for determining if and when surgical removal of pre-malignant precursor lesions (that have not yet developed into invasive cancer) should and should not be undertaken for an individual subject. For example, individuals with values of one or more biomarkers (MUC5AC glycan and CA 19-9 levels) different from a statistically validated threshold, or that are in the higher tertile or quartile of a “normal range,” could be identified as those in need of such surgical removal. Such medical treatments are known in the art.

Also included are methods of treatment of a patient having a pancreatic cystic lesion. The steps to implement this method are: obtaining a pancreatic cyst fluid sample from a pancreatic cystic lesion in a patient, assaying the sample for a glycan level of MUC5AC; determining whether the glycan level of MUC5AC in the sample is present at a higher level than the glycan level of MUC5AC in pancreatic cystic lesions having no malignant potential; and surgically removing the pancreatic cystic lesion from the patient if the glycan level of MUC5AC in the sample is present at the higher level.

With this method of treatment, the glycan level of MUC5AC may be assayed with a lectin, the lectin may be Vicia villosa, Jacalin, wheat-germ agglutinin (WGA), and Erythrina cristagalli lectin (ECL), and the sample may be obtained by endoscopic ultrasound fine-needle aspiration. Further, this method also may include: assaying the sample for a level of CA 19-9; determining whether the level of CA 19-9 in the sample is present at a higher level than the level of CA 19-9 in pancreatic cystic lesions having no malignant potential; and surgically removing the pancreatic cystic lesion from the patient if the glycan level of MUC5AC in the sample is present at a higher level than the glycan level of MUC5AC in pancreatic cystic lesions having no malignant potential and the level of CA 19-9 in the sample is present at a higher level than the level of CA 19-9 in pancreatic cystic lesions having no malignant potential.

Another aspect of the present invention is a method for treating a pancreatic cystic lesion in a patient comprising: obtaining a pancreatic cyst fluid sample from a patient having or suspected of having a pancreatic disease; assaying the sample for a glycan level of MUC5AC in the sample; comparing the glycan level in MUC5AC in the sample to a statistically validated threshold for MUC5AC, which statistically validated threshold for MUC5AC is based on a glycan level in MUC5AC in comparable control samples from benign pancreatic cysts in subjects; and surgically removing the pancreatic cystic lesion from the patient if the glycan level in MUC5AC in the sample is different than the statistically validated threshold. With this method, the glycan level of MUC5AC may be assayed with a lectin, the lectin may be Vicia villosa, Jacalin, wheat-germ agglutinin (WGA), and Erythrina cristagalli lectin (ECL), and the sample may be obtained by endoscopic ultrasound fine-needle aspiration. Additionally, this method may include: assaying the sample for a level of CA 19-9; comparing the glycan level of CA 19-9 in the sample to a statistically validated threshold for CA 19-9, which statistically validated threshold for CA 19-9 is based on a level of CA 19-9 in comparable control samples from benign pancreatic cysts in subjects; and surgically removing the pancreatic cystic lesion from the patient if the glycan level in MUC5AC in the sample is different than the statistically validated threshold for MUC5AC, and if the level of CA 19-9 in the sample is different than the statistically validated threshold for CA 19-9.

Having now generally described the invention, the same will be more readily understood through reference to the following examples, which are provided by way of illustration, and are not intended to be limiting of the present invention, unless specified.

### EXAMPLES

### Example 1

### Materials and Methods for Examples 2-4

Patients and Cyst Fluid Samples. The study was conducted in strict compliance with the guidelines of the University of Michigan and University of Indiana School of Medicine Institutional Review Boards. After obtaining signed informed consent, cyst fluid samples were collected at the time of endoscopy or operation at the University of Michigan (n=37) and Indiana University (n=16). All patients enrolled in the study had operative treatment of the cystic lesion and the surgical pathology report was used to confirm the diagnosis of cyst type in all patients. The majority of the IPMN lesions studied were side-branch lesions. Cyst fluid specimens were immediately placed on ice after procurement and were aliquoted and stored at −80° C. Each sample was thawed no more than three times prior to analysis in order to minimize variability introduced by that process. Each sample was centrifuged at 10,000×g for 10 minutes to remove remaining debris prior to use. The samples were randomized in their handling and experimental processing.

Biological reagents. Antibodies were purchased from various sources (see Table 1).

TABLE 1
Antibodies and Lectins Used for Biomarker Analysis

| Antibody or Lectin Name | Clone ID | Supplier | Catalog Number | Target |
|---|---|---|---|---|
| Anti-Alpha-1-antitrypsin (Ab 1) | Polyclonal | Abcam | AB7633 | Alpha-1-antitrypsin Core Protein |
| Anti-Alpha-1-antitrypsin (Ab 2) | 8A0 | Biotrend | BT06-4055-07 | Alpha-1-antitrypsin Core Protein |
| Anti-Amylase | O.G.3 | US Biological | A2274-05 | Amylase Core Protein |
| Anti-Angiostatin | 79735 | Sigma | A0976 | Angiostatin Core Protein |
| Anti-CA 19-9 | 1.B.844 | US Biological | C0075-07 | Sialyl Lewis A |
| Anti-CEA (Ab 1) | 2Q397 | US Biological | C1299-94 | CEA Core Protein |
| Anti-CEA (Ab 2) | 6D308 | US Biological | C2589-76A | CEA Core Protein |
| Anti-CEACAM6 | By114 | Santa Cruz | SC-20059 | CEACAM6 Core Protein |
| Anti-Endostatin | 1837-46 | Abcam | AB15685 | Endostatin Core Protein |
| Anti-Fibronectin | Polyclonal | R&D Systems | AF1918 | Fibronectin Core Protein |
| Anti-MUC1 (Ab 1) | 1.B.831 | US Biological | C0050-23 | MUC1 Core Protein |
| Anti-MUC1 (Ab 2) | SM3 | Abcam | AB22711 | MUC1 Core Protein |
| Anti-MUC16 (Ab 1) | 1.B.821 | US Biological | C0050-01 | MUC16 Core Protein |
| Anti-MUC16 (Ab 2) | X325 | Abcam | AB10033 | MUC16 Core Protein |
| Anti-MUC5AC (Ab 1) | 45M1 | Biogenesis | 1695-0128 | MUC5AC Core Protein |
| Anti-MUC5AC (Ab 2) | CLH2 | Chemicon International | MAB2011 | MUC5AC Core Protein |
| Anti-pan CEACAM | D14HD11 | Abcam | AB4567 | CEACAM1, 3, 4, 5, 6 |
| Goat IgG | N/A | Jackson Immunoresearch | 005-000-003 | N/A |
| Mouse IgG | N/A | Jackson Immunoresearch | 015-000-003 | N/A |
| Sheep IgG | N/A | Jackson Immunoresearch | 013-000-003 | N/A |
| Aleria aurantia Lectin (AAL) | N/A | Vector Labs | B-1395 | Alpha-linked fucose |
| Sambucus nigra lectin (SNA) | N/A | Vector Labs | B-1305 | Alpha-2,6-linked sialic acid |
| Concanavalin A (ConA) | N/A | Vector Labs | B-1005 | Alpha-linked mannose |
| Vicia villosa lectin (VVL) | N/A | Vector Labs | B-1235 | Alpha- or beta-linked terminal |
| | | | | N-acetylgalactosamine (GalNAc) |
| Wheat Germ Agglutinin (WGA) | N/A | Vector Labs | B-1025 | N-acetylglucosamine (GlcNAc) |
| Erythrina cristagalli lectin | N/A | Vector Labs | B-1145 | Galactosyl (β-1,4) N-acetylglucosamine |
| (ECL) | | | | (LacNAc) |
| Jacalin | N/A | Vector Labs | B-1155 | Galactosyl (β-1,3) N-acetylgalactosamine |
| | | | | (T-antigen) |

Antibodies were purified by dialysis (Slide-A-lyzer, Pierce Biotechnology, Rockford, Ill.) to PBS buffer and ultracentrifuged before the concentration of each antibody was adjusted to 500 μg/ml for microarray printing. The integrity and purity of each antibody was confirmed by SDS-PAGE under reducing and non-reducing conditions. Antibody biotinylation was performed using EZ-Link-sulfo-NHS-LC-biotin (Pierce Biotechnology, Rockford, Ill.).

Microarray fabrication and preparation. Antibody microarrays were prepared as previously described³¹. A piezoelectric non-contact printer (Biochip Arrayer, PerkinElmer Life Sciences, Waltham, Mass.) was used to spot approximately 350 pl of each antibody solution on the surfaces of ultrathin nitrocellulose-coated glass microscope slides (PATH slides, GenTel Biosciences, Madison, Wis.). Sixty identical arrays were printed on each slide, with each array consisting of 16 antibodies targeting proteins of interest, as well as control immunoglobulins from several species, printed in triplicate. A wax border was imprinted around each of the arrays to define hydrophobic boundaries (SlideImprinter, The Gel Company, San Francisco, Calif.). The printed slides were stored at 4° C. in a desiccated, vacuum-sealed slide box until use.

Sandwich assays. Assays were performed similar to previously described methods^{29,31}. Cyst fluid samples were diluted with PBS buffer containing 0.1% Brij, 0.1% Tween-20 and 50 ug/ml of protease inhibitor. An IgG/IgY cocktail consisting of a final concentration of 400 ug/ml goat, mouse and sheep IgG, 400 ug/ml chicken IgY and 800 ug/ml rabbit IgG (Jackson Immunoresearch, West Grove, Pa.) was added to each cyst fluid sample to eliminate non-specific binding to the printed antibodies. Slides were blocked in solution containing PBS-0.5% Tween-20 buffer (PBST0.5) with the addition of 1% BSA. Additionally, samples were spun at 16,000×g for 3 minutes to separate viscous components. 6 μl of sample was then applied to each array. Captured antigens were detected with biotinylated antibodies at a concentration of 1-10 μg/ml, followed by incubation with 1 μg/ml streptavidin-phycoerythrin (Roche Applied Science, Indianapolis, Ind.). The slides were scanned for fluorescence emission at 575 nm using a microarray scanner (LS Reloaded, TECAN, Durham, N.C.). All arrays assaying the same glycoprotein were scanned concurrently at a single laser power and detector gain setting.

Lectin detection assays. Prior to using the antibody microarrays for glycan detection, the glycans on the spotted antibodies were chemically derivatized as described previously²⁹ to prevent lectin binding to the capture antibodies. Briefly, the slides were incubated in a coupling buffer (0.1M sodium acetate, pH5.5, with 0.1% Tween-20) for 30 min, transferred into 200 mM NaIO4 solution (Pierce Biotechnology, Rockford, Ill.) and incubated at 4° C. for 2 hr in the dark to oxidize the sugar groups on printed antibodies. The slides were rinsed in coupling buffer and incubated with a solution containing 1 mM MPBH (4-(4-N-Maleimidophenyl))butyric acid hydrazide hydrochloride (Pierce Biotechnology, Rockford, Ill.) and 1 mM Cys-Gly dipeptide (Sigma-Aldrich, St. Louis, Mo.) for 2 hr at room temperature to derivatize the carbonyl groups. The slides were rinsed with PBST0.1 buffer and incubated with 1 mM Cys-Gly in PBST0.1 buffer overnight at 4° C. After the slides were rinsed thoroughly with PBST0.1 buffer and dried by centrifugation, the sandwich assay protocol was followed. Instead of detection antibodies, biotin-labeled lectins were used as the detection reagent at a concentration of 10 ug/ml.

Western blots. Cyst fluid samples were diluted 1:10 in sample buffer (final concentrations of 50 mM Tris, pH 6.8; 2% SDS; 10% glycerol; 2.5% beta-Mercaptoethanol; and 0.02% Bromophenol blue), and cell lysates were diluted 1:1.5 in sample buffer. Fifteen μl of each diluted sample was boiled for five minutes, separated on 4-12% Bis-Tris SDS-PAGE gels (Criterion XT, Bio-Rad, Hercules, Calif.), and transferred to a nitrocellulose membrane (0.45 μM, BioRad). The membranes were blocked overnight, probed with monoclonal antibodies or biotinylated lectins, and detected with anti-mouse IgG-HRP or streptavidin conjugated to HRP, respectively (Pierce Biotechnology, Rockford, Ill.). The stained bands were visualized using the SuperSignal West Pico Chemiluminescent substrate (Pierce Biotechnology, Rockford, Ill.), followed by exposure to Kodak Biomax XAR film (Sigma-Aldrich, St. Louis, Mo.).

Data analysis. Image data were quantified using GenePix Pro 5.1 (Axon Instruments, Union City, Calif.). The net fluorescent signal was calculated by subtracting the median local background surrounding each spot from the median intensity of the corresponding spot. The signal intensities from replicate antibody measurements within the same array were averaged.

Some measurements were removed from the analysis due to high signal in the negative controls, indicating possible high levels of non-specific binding. For a given lectin-antibody combination, if the average signal over all the samples was not greater than twice the signal detected at the mouse IgG negative control spots (using the relevant detection lectin), or if the average signal was not greater than twice the signal detected from PBS as the sample instead of cyst fluid (using the relevant lectin-antibody combination), then the given lectin-antibody combination was removed from subsequent analyses.

Multiparametric classification. The logistic regression forward selection with 5-fold cross-validation (CV) was implemented for multiparametric model building. At each iteration, the classifier was selected so that it gave the biggest incremental value among all the remaining antibodies to the likelihood of the existing marker panel. The coefficients of the classifiers were updated correspondingly. The inventors used a cross-validation process to determine the optimal number of antibodies in the marker panel. Due to small sample size, a 5-fold cross-validation was applied in this study, where 80% of the samples were used as training set to define a best model for classification while 20% of the samples were reserved as testing set to determine the error rate of the model when using prediction probability 0.5 as cut-off point. This process was repeated 5 times, each time using a different group of 80% for classification, and the cross-validation error is the average of the five error rates. The entire 5-fold CV was then repeated 100 times and a classifier is considered final when the further addition of an antibody caused an increase in the averaged cross-validation error. The cross-validation process simulates the uncertainty in the classification algorithm and estimates the prediction error of the selected combined classifier. Therefore, this validation gives extra protection against the chance of over-fitting, or creating a classifier specifically for a particular sample set.

### Example 2

### Profiling Protein and Glycan Levels in Cyst Fluid Samples

Cyst fluid samples were collected from 53 different patients (Table 2), and the samples were grouped according to the pathological examination of the resected cyst, including serous cystadenoma (n=12), pseudocyst (n=9), mucinous cystic neoplasm (n=17), and intraductal papillary mucinous neoplasm (n=15).

TABLE 2
Patient Demographic Information.

| | | | Average Age |
|---|---|---|---|
| Type | Number | Male (%) | (std. dev.) |
| PC | 9 | 2 (22%) | 35 (17) |
| SC | 12 | 4 (33%) | 43 (18) |
| MCN | 17 | 0 (0%) | 46 (12) |
| IPMN | 15 | 6 (40%) | 63 (13) |
| Total | 53 | 12 (23%) | 48 (17) |
| p-value, (PC + SC) vs. | | 0.4 | 0.002 |
| (MCN + IPMN) | | | |

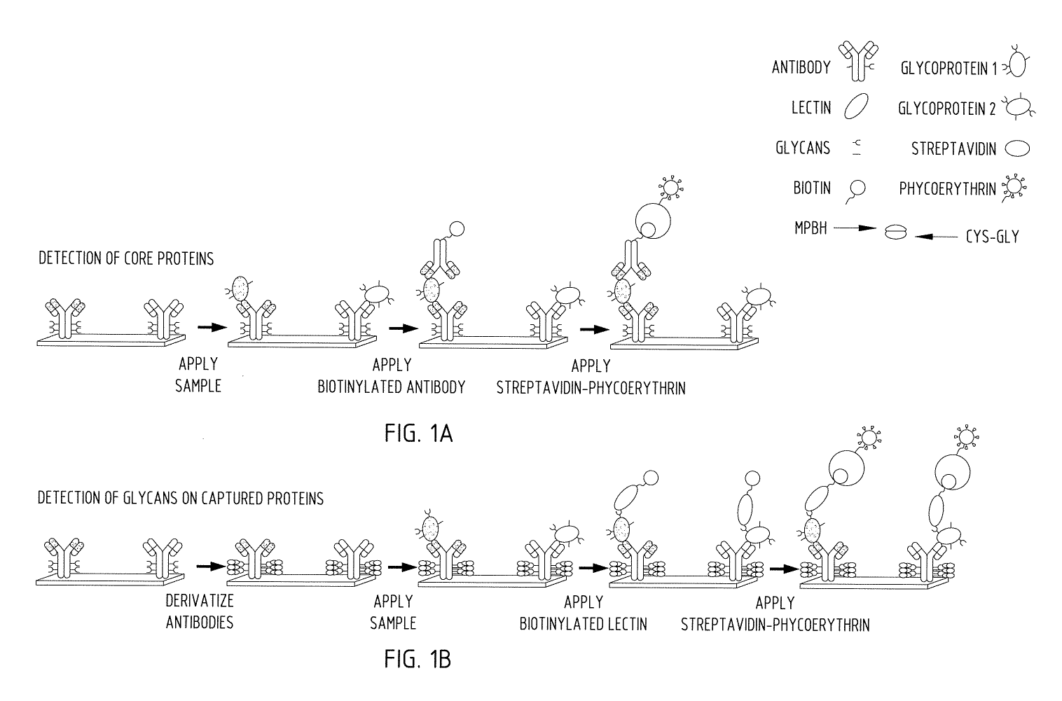

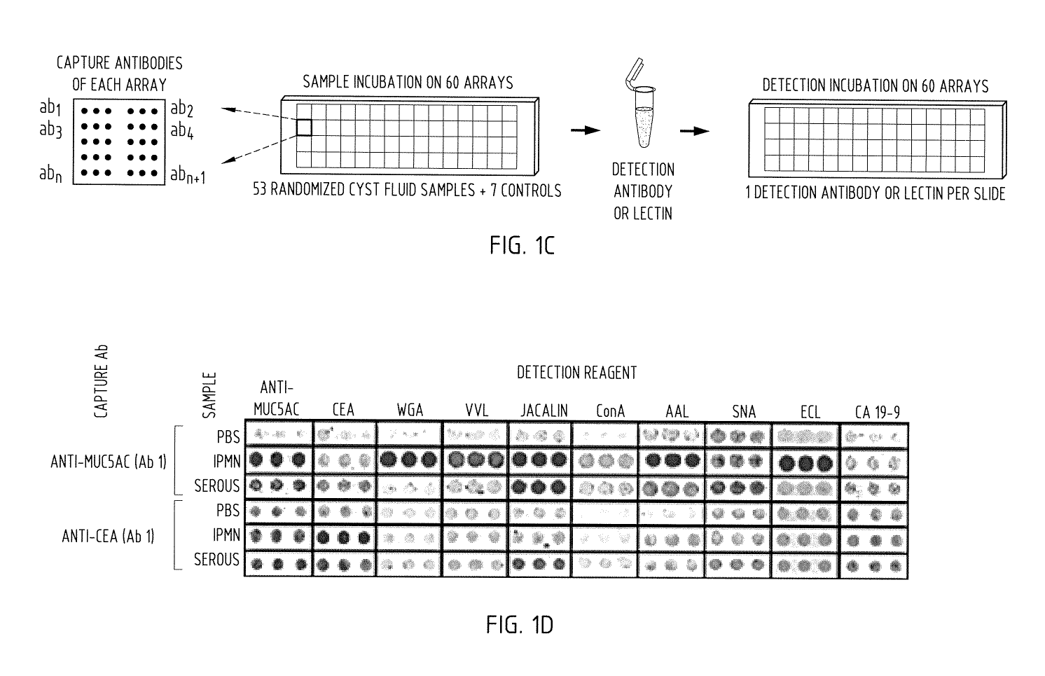

The samples were analyzed using a novel format of antibody microarray that makes it possible to obtain measurements of protein levels and their associated glycans in parallel assays (FIGS. 1A and 1B). Using small sample volumes (≦3 μl of cyst fluid diluted to 6 μl), samples were incubated on antibody arrays to allow the capture of multiple, specific proteins. The levels of the core proteins were probed with the appropriate antibodies (FIG. 1A), and the glycan levels on the captured proteins were probed with a variety of lectins (FIG. 1B). The ability to process microarrays in a high-throughput mode (FIG. 1C) allowed the probing of many samples with many different detection reagents, while the low volume of each assay enabled the probing of each sample many times. Representative data shows the detection of proteins by antibodies and their glycans using lectins (FIG. 1D). Each lectin binds a distinct glycan structure (Table 1), and the variation between proteins and samples in the levels of particular glycan structures is observed in the lectin binding patterns.

### Example 3

### Discriminating Patient Groups

In order to address the diagnostic challenge of discriminating mucin-producing neoplasms (MCN+IPMN) from benign cystic lesions (SC+PC), the inventors quantified the relative protein and glycan levels in individual cyst fluid samples and then compared the levels between the groups. Multiple protein and glycan measurements were significantly different (p<0.02) between mucin-producing and benign lesions (Table 3).

TABLE 3
Markers that significantly discriminate mucinous from non-mucinous cysts

| Capture Antibody | Detection Antibody or Lectin | p values | AUC (95% C.I.) | Sens. at 80% spec. | Spec. at 80% sens. | Up-regulated in |
|---|---|---|---|---|---|---|
| Anti-MUC5AC Ab 1 | Anti-MUC5AC Ab 1 | 0.0090 | 0.72 (0.56-0.84) | 0.53 | 0.43 | Mucinous |
| Anti-MUC5AC Ab 1 | WGA | 0.0015 | 0.88 (0.76-0.95) | 0.78 | 0.76 | Mucinous |
| Anti-MUC5AC Ab 1 | Jacalin | 4.00E−04 | 0.83 (0.73-0.93) | 0.72 | 0.7 | Mucinous |
| Anti-MUC5AC Ab 1 | VVL | 0.0140 | 0.82 (0.68-0.93) | 0.75 | 0.52 | Mucinous |
| Anti-MUC5AC Ab 1 | ECL | 0.0018 | 0.81 (0.64-0.88) | 0.69 | 0.57 | Mucinous |
| Anti-CEACAM6 | Anti-pan CEACAM | NS (0.83) | — | — | — | — |
| Anti-CEACAM6 | ECL | 0.010 | 0.70 (0.57-0.86) | 0.5 | 0.52 | Nonmucinous |
| Anti-CEACAM6 | ConA | 0.0096 | 0.67 (0.53-0.81) | 0.56 | 0.38 | Nonmucinous |
| Anti-CA 19-9 | Anti-CA 19-9 | 2.00E−04 | 0.79 (0.66-0.90) | 0.75 | 0.24 | Mucinous |
| Anti-CEA Ab 1 | Anti-pan CEACAM | NS (0.025) | 0.67 (0.55-0.83) | 0.37 | 0.67 | — |
| Anti-CEA Ab 1 | Anti-CA 19-9 | 0.018 | 0.84 (0.73-0.92) | 0.72 | 0.71 | Mucinous |
| Anti-MUC1 Ab 1 | Anti-MUC1 Ab 1 | 0.0093 | 0.64 (0.47-0.80) | 0.19 | 0.48 | Nonmucinous |
| Anti-Fibronectin | Jacalin | 0.013 | 0.69 (0.56-0.82) | 0.5 | 0.43 | Nonmucinous |

In Table 3, assays are shown that have p<0.02 and that passed criteria (described in Example 1 above) to eliminate measurements with possible high contributions from non-specific binding. AUC, area-under-the-curve; C.I., confidence interval.

Specifically, MUC5AC and its glycan variants were elevated in the mucin-producing cystic tumor samples, while MUC1 was elevated in the non-mucin-producing samples. CEA was moderately elevated in the mucinous samples (p=0.025), and glycan variants of CEACAM6 were elevated in the non-mucinous samples. The CA 19-9 antigen was higher in the mucin-producing cystic tumor samples.

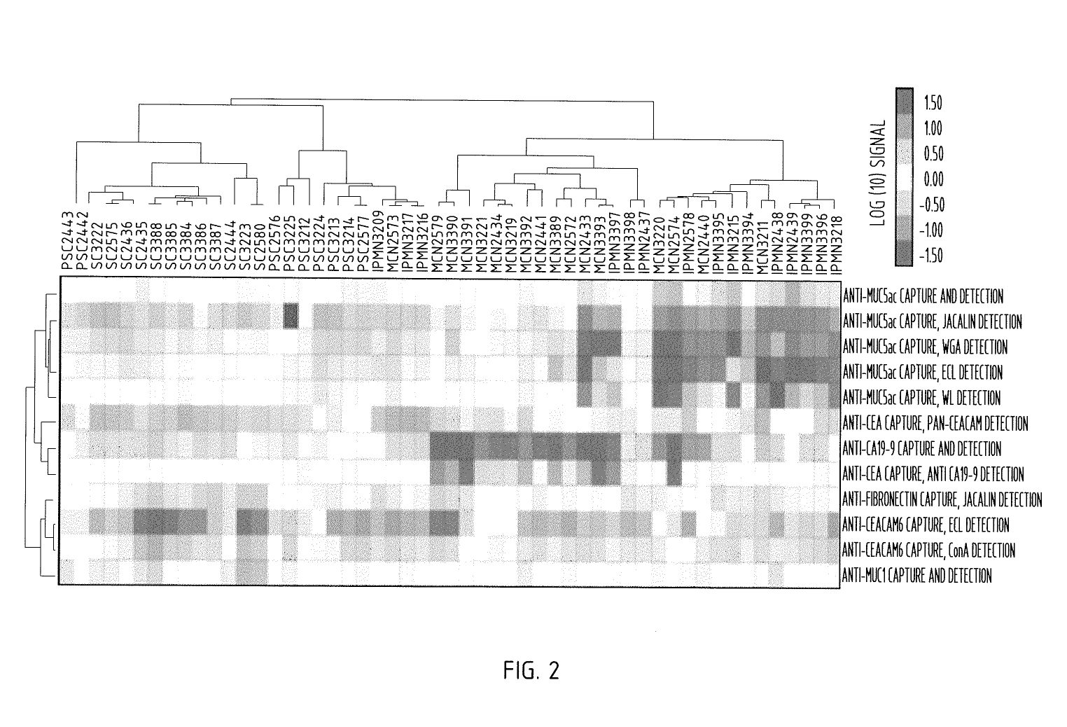

A cluster of the most significant measurements (p<0.02) shows the patterns among the different patient samples (FIG. 2). The samples clearly segregate according to their status as either mucin-producing cystic tumors or benign cystic lesions (serous cystadenomas and pseudocysts). Subgroups within those classifications were also evident, as the serous cystadenomas are segregated from pseudocysts, and many of the mucin-producing cystic tumors had divergent expression patterns of CA 19-9 and MUC5AC.

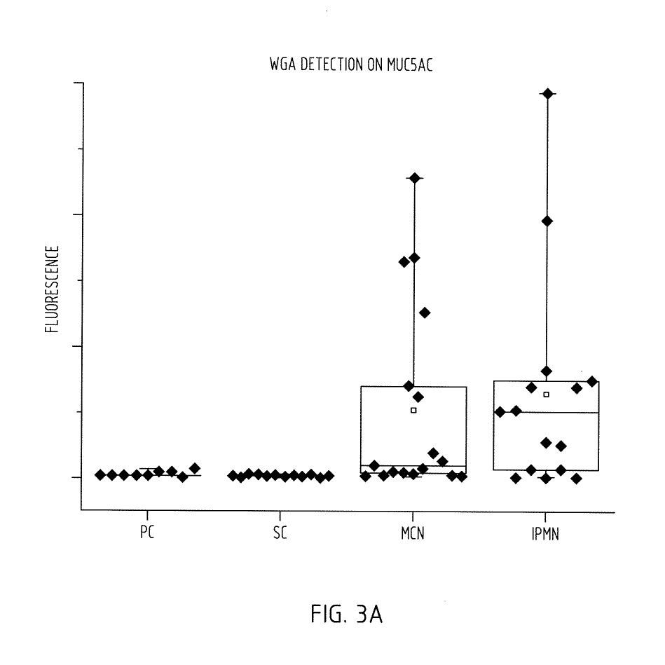

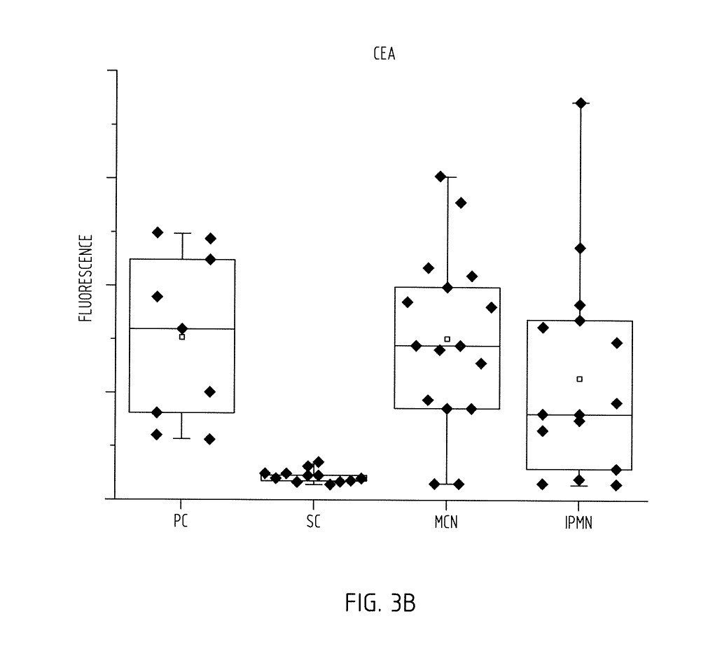

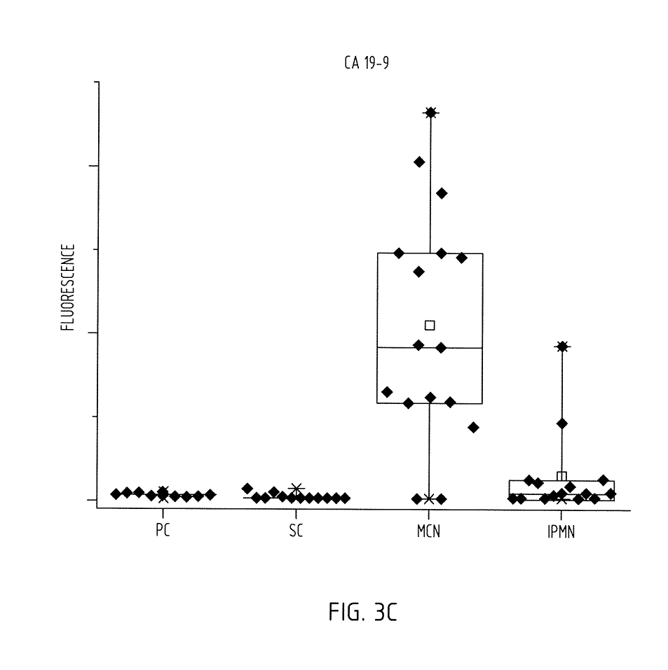

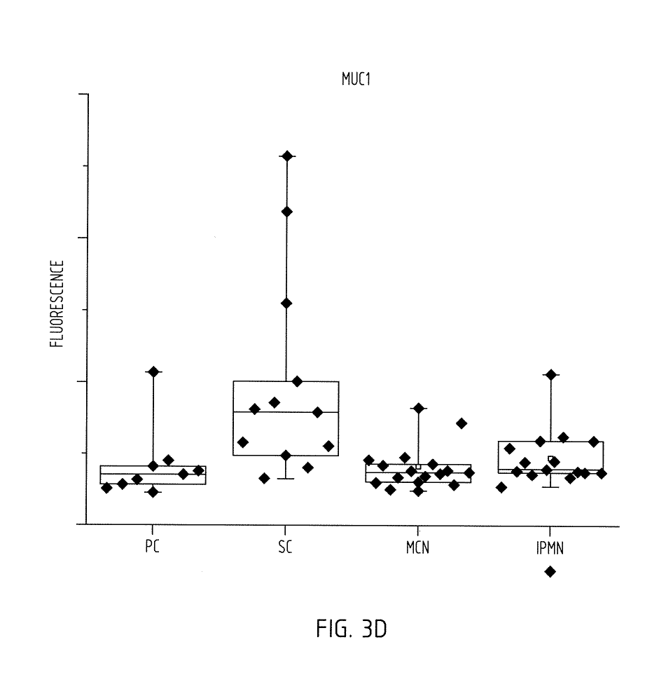

The inventors examined in more detail the biomarkers with the greatest differences between the groups. One of the most significant biomarkers was Wheat Germ Agglutinin (WGA) detection of the MUC5AC protein (indicated as WGA-MUC5AC). WGA-MUC5AC showed no elevation in PC and SC but high elevation in some of the MCN and IPMN samples (FIG. 3A), indicating that a biomarker based on this molecule may have good specificity (low false positives) and moderate sensitivity (moderate true positives) for distinguishing mucin-producing from non-mucinous benign lesions (78% sensitivity at 80% specificity). CEA was elevated in a higher proportion of the mucin-producing cystic tumors, and was not elevated in the serous cystadenomas, but it was elevated in the PC, yielding good performance in distinguishing SC from mucin-producing cystic tumors but only moderate performance distinguishing mucin-producing cystic tumors from all non-mucinous cysts (37% sensitivity at 80% specificity) (FIG. 3B). CA 19-9 was primarily elevated in MCN, rarely in IPMN, and not at all in the non-mucinous cysts (FIG. 3C). MUC1, in contrast to its related mucin family member MUC5AC, was elevated primarily in the serous cystadenomas (FIG. 3D). The complementary nature of these markers suggests that some markers may be used in combination to achieve higher-accuracy discrimination as compared to the individual markers. The discrimination of MCN from IPMN also can be diagnostically challenging in some patients. The best biomarker to discriminate between MCN and IPMN was CA 19-9 (sensitivity/specificity=82%/93%) (FIG. 3C). No other marker performed nearly as well for distinguishing MCN from IPMN.

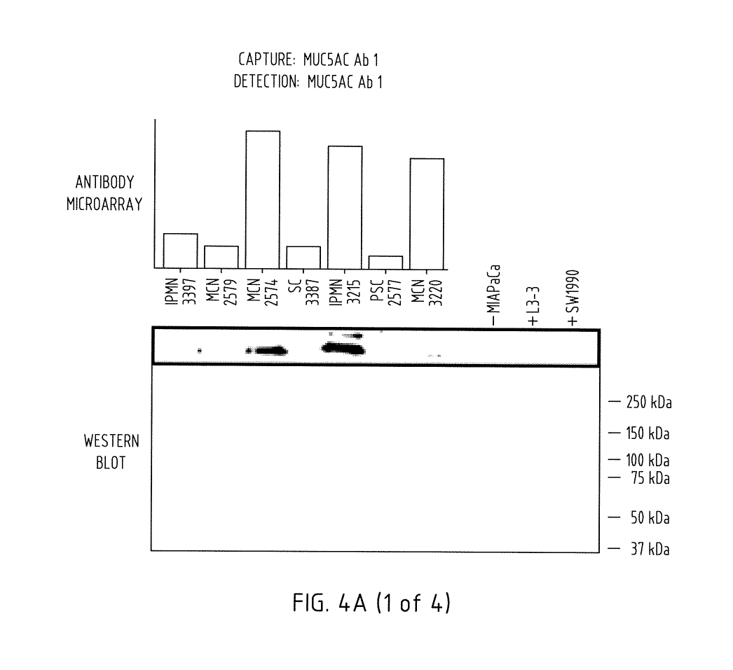

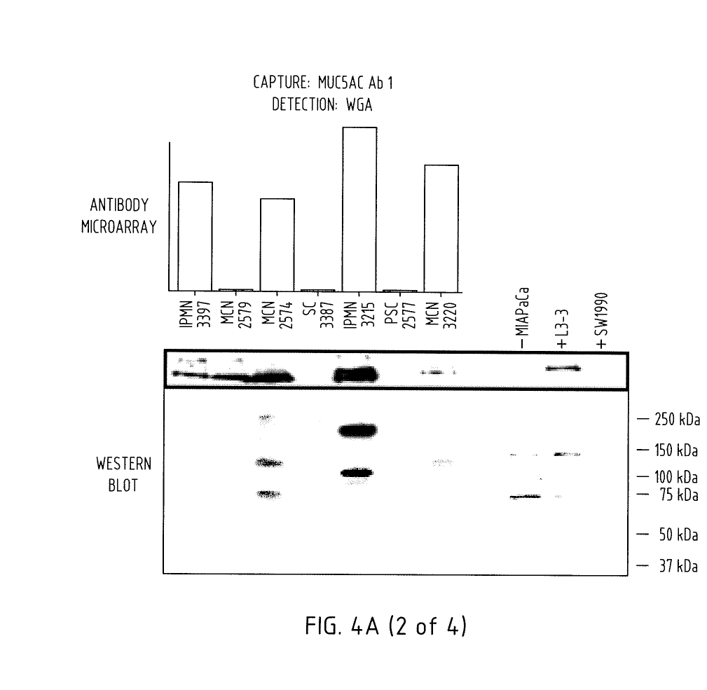

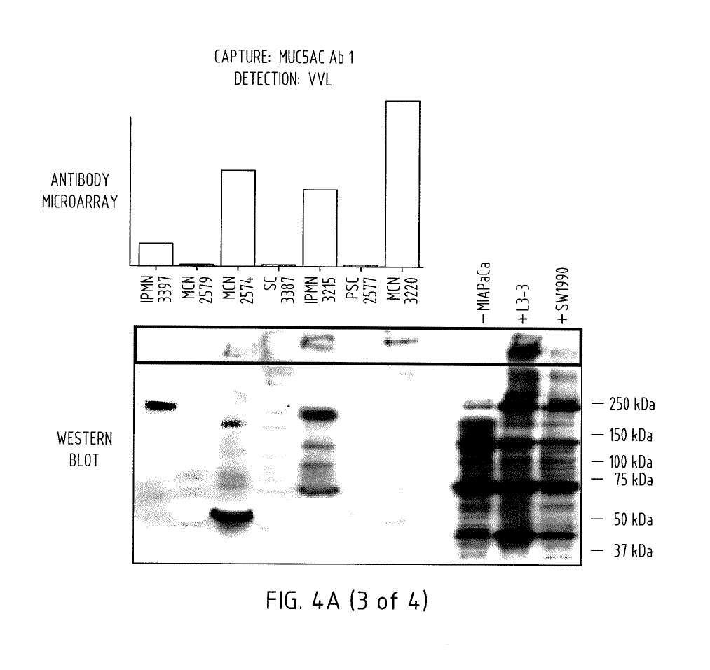

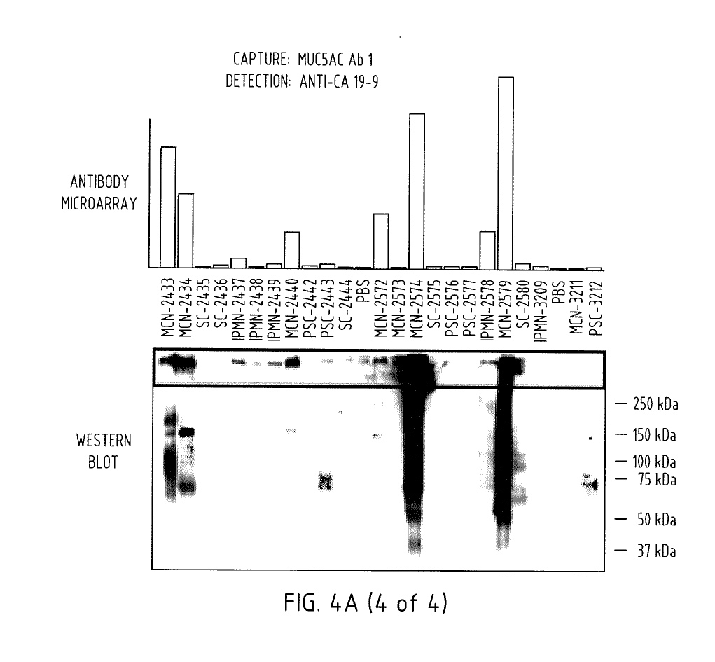

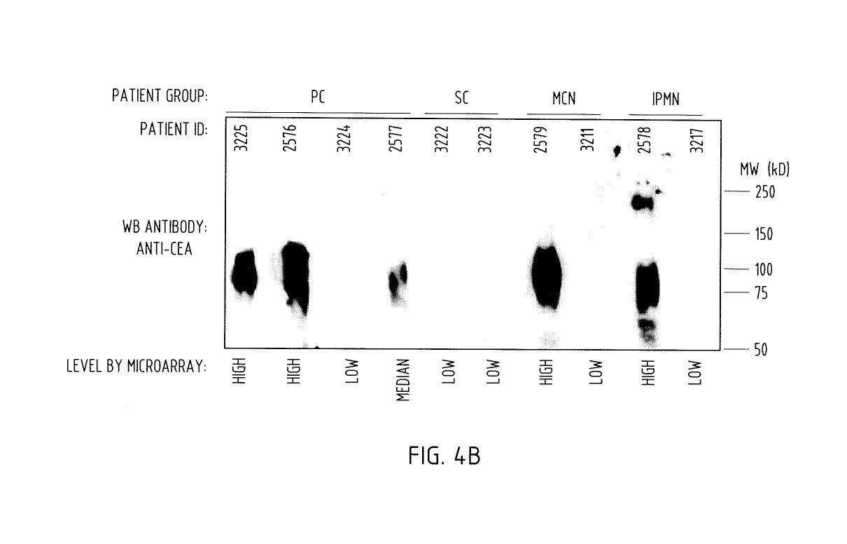

The expression patterns of certain biomarkers were examined by Western blotting as a validation of the accuracy of the microarray measurements. Western blots detection the MUC5AC protein confirmed the protein levels detected by antibody microarray, and lectin blots of the same serum samples showed higher signal at overlapping bands, which supports the presence of particular glycans on MUC5AC (FIG. 4A). Other bands detected by the lectins that are not overlapping with the MUC5AC bands could be due to other proteins carrying the relevant glycan, or splice variants of MUC5AC. Western blots for CEA levels also confirmed the accuracy of the antibody microarray measurements for that protein (FIG. 4B).

### Example 4

### Complementary Marker Patterns

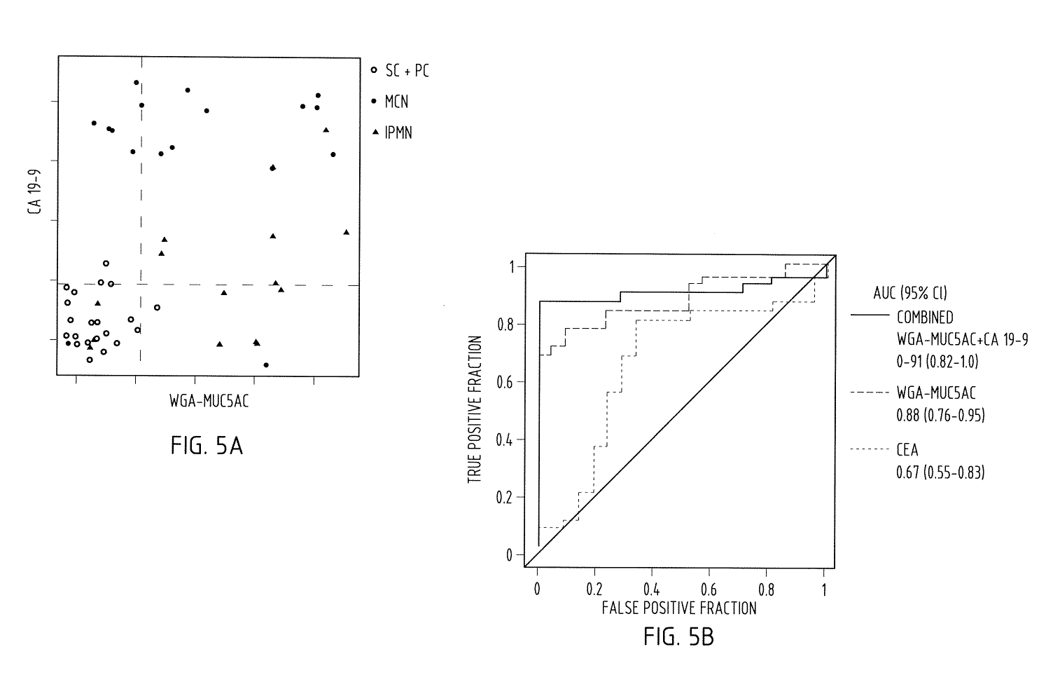

The inventors examined the possibility of using combinations of two or more biomarkers to improve the accuracy of discriminating different patient groups relative to the individual biomarkers. A requirement for such an outcome is that the individual markers provide complementary information, which is suggested by the distinct patterns of markers of FIG. 2. An effective method for testing the performance of combinations of markers is linear regression with forward selection³¹⁻³⁴. This method was applied to the discrimination of mucin-producing cystic tumors (MCN+IPMN) from non-mucinous cystic lesions (SC+PC) using the entire data set. The method found that CA 19-9 and WGA-MUC5AC provide highly complementary information, with CA 19-9 identifying most of the MCNs, WGA-MUC5AC identifying most of the IPMNs, and neither showing frequent elevation in the non-mucinous cysts (FIG. 5A). Based on that relationship, a classification algorithm using both biomarkers gave an area-under-the-curve (AUC) in receiver-operator-characteristic (ROC) analysis of 0.91, and a sensitivity of 87% at 86% specificity. This performance was better than either WGA-MUC5AC (0.88 AUC, 78%/80% sensitivity/specificity) or CA 19-9 (0.79 AUC, 75%/80% sensitivity/specificity) used individually (FIG. 5B). The improvement was highly significant relative to CA 19-9 alone (p=0.02 in the comparison of the markers) but not relative to WGA-MUC5AC alone, although the improvement may be significant using greater sample numbers. The combined marker performed significantly better than CEA (p<0.001 in the comparison of the markers), which gave a 0.67 AUC and a 37%/80% sensitivity/specificity.

While the foregoing specification has been described with regard to certain preferred embodiments, and many details have been set forth for the purpose of illustration, it will be apparent to those skilled in the art that the invention may be subject to various modifications and additional embodiments, and that certain of the details described herein can be varied considerably without departing from the spirit and scope of the invention. Such modifications, equivalent variations and additional embodiments are also intended to fall within the scope of the appended claims.

### REFERENCES

1. Sohn T A, Yeo C J, Cameron J L, et al. Resected adenocarcinoma of the pancreas-616 patients: results, outcomes, and prognostic indicators. J Gastrointest Surg 2000; 4(6):567-79.
2. Singh M, Maitra A. Precursor lesions of pancreatic cancer: molecular pathology and clinical implications. Pancreatology 2007; 7(1):9-19.
3. Canto M I, Goggins M, Hruban R H, et al. Screening for early pancreatic neoplasia in high-risk individuals: a prospective controlled study. Clin Gastroenterol Hepatol 2006; 4(6):766-81; quiz 665.
4. Wilentz R E, Albores-Saavedra J, Zahurak M, et al. Pathologic examination accurately predicts prognosis in mucinous cystic neoplasms of the pancreas. Am J Surg Pathol 1999; 23(11):1320-7.
5. Hruban R H, Goggins M, Parsons J, et al. Progression model for pancreatic cancer. Clin Cancer Res 2000; 6(8):2969-72.
6. Hruban R H, Takaori K, Klimstra D S, et al. An illustrated consensus on the classification of pancreatic intraepithelial neoplasia and intraductal papillary mucinous neoplasms. Am J Surg Pathol 2004; 28(8):977-87.
7. Adsay N V. Cystic lesions of the pancreas. Modern Pathology 2007; 20:S71-S93.
8. Winter J M, Cameron J L, Lillemoe K D, et al. Periampullary and pancreatic incidentaloma: a single institution's experience with an increasingly common diagnosis. Ann Surg 2006; 243(5):673-80; discussion 680-3.
9. Spinelli K S, Fromwiller T E, Daniel R A, et al. Cystic pancreatic neoplasms: observe or operate. Ann Surg 2004; 239(5):651-7; discussion 657-9.
10. Brugge W R, Lewandrowski K, Lee-Lewandrowski E, et al. Diagnosis of pancreatic cystic neoplasms: a report of the cooperative pancreatic cyst study. Gastroenterology 2004; 126(5):1330-6.
11. van der Waaij L A, van Dullemen H M, Porte R J. Cyst fluid analysis in the differential diagnosis of pancreatic cystic lesions: a pooled analysis. Gastrointest Endosc 2005; 62(3):383-9.
12. Khalid A, McGrath K M, Zahid M, et al. The role of pancreatic cyst fluid molecular analysis in predicting cyst pathology. Clin Gastroenterol Hepatol 2005; 3(10):967-73.
13. Raty S, Sand J, Alfthan H, et al. Cyst fluid tumor-associated trypsin inhibitor may be helpful in the differentiation of cystic pancreatic lesions. J Gastrointest Surg 2004; 8(5):569-74.
14. Shami V M, Sundaram V, Stelow E B, et al. The level of carcinoembryonic antigen and the presence of mucin as predictors of cystic pancreatic mucinous neoplasia. Pancreas 2007; 34(4):466-9.
15. Hammel P R, Forgue-Lafitte M E, Levy P, et al. Detection of gastric mucins (M1 antigens) in cyst fluid for the diagnosis of cystic lesions of the pancreas. Int J Cancer 1997; 74(3):286-90.
16. Anderson M A, Kwon R S, Scheiman J M. PANDA cyst fluid analysis: eats, shoots and leaves? Gastrointest Endosc 2009 (in press).
17. Burdick M D, Harris A, Reid C J, et al. Oligosaccharides expressed on MUC1 produced by pancreatic and colon tumor cell lines. J Biol Chem 1997; 272(39):24198-202.
18. Moniaux N, Andrianifahanana M, Brand R E, et al. Multiple roles of mucins in pancreatic cancer, a lethal and challenging malignancy. Br J Cancer 2004; 91(9):1633-8.
19. Hollingsworth M A, Swanson B J. Mucins in cancer: protection and control of the cell surface. Nat Rev Cancer 2004; 4(1):45-60.
20. Dennis J W, Granovsky M, Warren C E. Glycoprotein glycosylation and cancer progression. Biochim Biophys Acta 1999; 1473(1):21-34.
21. Thompson S, Cantwell B M, Cornell C, et al. Abnormally-fucosylated haptoglobin: a cancer marker for tumour burden but not gross liver metastasis. Br J Cancer 1991; 64(2):386-90.
22. Okuyama N, Ide Y, Nakano M, et al. Fucosylated haptoglobin is a novel marker for pancreatic cancer: A detailed analysis of the oligosaccharide structure and a possible mechanism for fucosylation. Int J Cancer 2005.
23. Miyoshi E, Nakano M. Fucosylated haptoglobin is a novel marker for pancreatic cancer: detailed analyses of oligosaccharide structures. Proteomics 2008; 8(16):3257-62.
24. Thompson S, Guthrie D, Turner G A. Fucosylated forms of alpha-l-antitrypsin that predict unresponsiveness to chemotherapy in ovarian cancer. Br J Cancer 1988; 58(5):589-93.
25. Mita Y, Aoyagi Y, Suda T, et al. Plasma fucosyltransferase activity in patients with hepatocellular carcinoma, with special reference to correlation with fucosylated species of alpha-fetoprotein. J Hepatol 2000; 32(6):946-54.
26. Naitoh A, Aoyagi Y, Asakura H. Highly enhanced fucosylation of serum glycoproteins in patients with hepatocellular carcinoma. J Gastroenterol Hepatol 1999; 14(5):436-45.
27. Li C, Simeone D M, Brenner D E, et al. Pancreatic Cancer Serum Detection Using a Lectin/Glyco-Antibody Array Method. J Proteome Res 2008.
28. Zhao J, Patwa T H, Qiu W, et al. Glycoprotein microarrays with multi-lectin detection: unique lectin binding patterns as a tool for classifying normal, chronic pancreatitis and pancreatic cancer sera. J Proteome Res 2007; 6(5):1864-74.
29. Chen S, LaRoche T, Hamelinck D, et al. Multiplexed analysis of glycan variation on native proteins captured by antibody microarrays. Nat Methods 2007; 4(5):437-44.
30. Chen S, Haab B B. Antibody Microarrays for Protein and Glycan Detection. In: Van Eyk J, Dunn M, eds. Clinical Proteomics. Weinheim, Germany: Wiley, VCH; 2007.
31. Orchekowski R, Hamelinck D, Li L, et al. Antibody microarray profiling reveals individual and combined serum proteins associated with pancreatic cancer. Cancer Res 2005; 65(23):11193-202.
32. Duh M S, Walker A M, Pagano M, et al. Prediction and cross-validation of neural networks versus logistic regression: using hepatic disorders as an example. Am J Epidemiol 1998; 147(4):407-13.
33. Hastie T, Tibshirani R, Friedman J. The Elements of Statistical Learning: Data Mining, Inference, and Prediction. New York: Springer; 2001.
34. Thall P, Simon R, Grier D. Test-based variable selection via cross-validation. Journal of Computational and Graphical Statistics 1992; 1:41-61.
35. Forrester S, Kuick R, Hung K E, et al. Low-volume, high-throughput sandwich immunoassays for profiling plasma proteins in mice: identification of early-stage systemic inflammation in a mouse model of intestinal cancer. Molecular Oncology 2007; 1:216-225.
36. Springer G F. Immunoreactive T and Tn epitopes in cancer diagnosis, prognosis, and immunotherapy. J Mol Med 1997; 75(8):594-602.
37. Brockhausen I. Mucin-type O-glycans in human colon and breast cancer: glycodynamics and functions. EMBO Rep 2006; 7(6):599-604.
38. Schuessler M H, Pintado S, Welt S, et al. Blood group and blood-group-related antigens in normal pancreas and pancreas cancer: enhanced expression of precursor type 1, Tn and sialyl-Tn in pancreas cancer. Int J Cancer 1991; 47(2):180-7.
39. Yu L G. The oncofetal Thomsen-Friedenreich carbohydrate antigen in cancer progression. Glycoconj J2007; 24(8):411-20.
40. Kobata A, Amano J. Altered glycosylation of proteins produced by malignant cells, and application for the diagnosis and immunotherapy of tumours. Immunol Cell Biol 2005; 83(4):429-39.
41. Saito H, Nishikawa A, Gu J, et al. cDNA cloning and chromosomal mapping of human N-acetylglucosaminyltransferase V+. Biochem Biophys Res Commun 1994; 198(1):318-27.
42. Shimodaira K, Nakayama J, Nakamura N, et al. Carcinoma-associated expression of core 2 beta-1,6-N-acetylglucosaminyltransferase gene in human colorectal cancer: role of O-glycans in tumor progression. Cancer Res 1997; 57(23):5201-6.
43. McEver R P. Selectin-carbohydrate interactions during inflammation and metastasis. Glycoconj J 1997; 14(5):585-91.
44. Kishimoto T, Ishikura H, Kimura C, et al. Phenotypes correlating to metastatic properties of pancreas adenocarcinoma in vivo: the importance of surface sialyl Lewis(a) antigen. Int J Cancer 1996; 69(4):290-4.
45. Iwai K, Ishikura H, Kaji M, et al. Importance of E-selectin (ELAM-1) and sialyl Lewis(a) in the adhesion of pancreatic carcinoma cells to activated endothelium. Int J Cancer 1993; 54(6):972-7.
46. Kawarada Y, Ishikura H, Kishimoto T, et al. The role of sialylated Lewis antigens on hematogenous metastases of human pancreas carcinoma cell lines in vivo. Pathol Res Pract 2000; 196(4):259-63.
47. Ferrone C R, Finkelstein D M, Thayer S P, et al. Perioperative CA19-9 levels can predict stage and survival in patients with resectable pancreatic adenocarcinoma. J Clin Oncol 2006; 24(18):2897-902.
48. Takahashi S, Oda T, Hasebe T, et al. Overexpression of sialyl Lewis x antigen is associated with formation of extratumoral venous invasion and predicts postoperative development of massive hepatic metastasis in cases with pancreatic ductal adenocarcinoma. Pathobiology 2001; 69(3):127-35.

What is claimed is:

1. A method of diagnosing a malignant potential of a pancreatic cystic lesion in a subject comprising:
obtaining a pancreatic cyst fluid sample from a pancreatic cystic lesion in a subject,
assaying the sample for a glycan level of MUC5AC in the sample,
determining whether the level of MUC5AC glycan is differentially present in the sample, and
diagnosing the malignant potential of the cystic lesion.

2. The method of claim 1, wherein it is determined that the glycan level of MUC5AC is differentially present at a higher level in the sample as compared to pancreatic cystic lesions having no malignant potential, and the cystic lesion is diagnosed as having malignant potential.

3. The method of claim 1, wherein the glycan level is detected by a lectin.

4. The method of claim 3, wherein the lectin is selected from the group consisting of Vicia villosa, Jacalin, wheat-germ agglutinin (WGA), and Erythrina cristagalli lectin (ECL).

5. The method of claim 1, wherein the sample is obtained by endoscopic ultrasound fine-needle aspiration.

6. The method of claim 1, further comprising:
assaying the sample for a CA 19-9 level in the sample,
determining whether the level of CA 19-9 is differentially present in the sample, and
diagnosing the malignant potential of the cystic lesion.

7. The method of claim 6, wherein it is determined that the glycan level of MUC5AC and the level of CA 19-9 are differentially present at higher levels in the sample as compared to pancreatic cystic lesions having no malignant potential, and the cystic lesion is diagnosed as having malignant potential.

8. A method for determining the malignant potential of a pancreatic cyst lesion, comprising the steps:
obtaining a pancreatic cyst fluid sample from a patient having or suspected of having a pancreatic disease;
assaying the sample for a glycan level of MUC5AC in the sample;
comparing the glycan level in MUC5AC in the sample to a statistically validated threshold for MUC5AC, which statistically validated threshold for MUC5AC is based on a glycan level in MUC5AC in comparable control samples from benign pancreatic cysts in subjects,
wherein a different glycan level in MUC5AC in the sample as compared to the statistically validated threshold indicates that the pancreatic cyst lesion from which the sample was obtained has malignant potential.

9. The method of claim 8, further comprising:
assaying the sample for a CA 19-9 level in the sample; and
comparing the level of CA 19-9 antigen in the sample to a statistically validated threshold for CA 19-9 antigen, which statistically validated threshold for CA 19-9 antigen is based on a level of CA 19-9 antigen in comparable control samples from benign pancreatic cysts in subjects;
wherein (a) a different level of CA 19-9 antigen in the sample as compared to the statistically validated threshold for CA 19-9 antigen and (b) a different level of glycan level in the MUC5AC in the sample as compared to the statistically validated threshold for the MUC5AC indicate that the pancreatic cyst lesion from which the sample was obtained has malignant potential.

10. A method for treating a pancreatic cystic lesion in a patient comprising:
obtaining a pancreatic cyst fluid sample from a pancreatic cystic lesion in a patient,
assaying the sample for a glycan level of MUC5AC
determining whether the glycan level of MUC5AC in the sample is present at a higher level than the glycan level of MUC5AC in pancreatic cystic lesions having no malignant potential; and
surgically removing the pancreatic cystic lesion from the patient if the glycan level of MUC5AC in the sample is present at the higher level.

11. The method of claim 10, wherein the glycan level of MUC5AC is assayed with a lectin.

12. The method of claim 11, wherein the lectin is selected from the group consisting of Vicia villosa, Jacalin, wheat-germ agglutinin (WGA), and Erythrina cristagalli lectin (ECL).

13. The method of claim 10, wherein the sample is obtained by endoscopic ultrasound fine-needle aspiration.

14. The method of claim 10, further comprising:
assaying the sample for a level of CA 19-9;
determining whether the level of CA 19-9 in the sample is present at a higher level than the level of CA 19-9 in pancreatic cystic lesions having no malignant potential; and
surgically removing the pancreatic cystic lesion from the patient if the glycan level of MUC5AC in the sample is present at a higher level than the glycan level of MUC5AC in pancreatic cystic lesions having no malignant potential and the level of CA 19-9 in the sample is present at a higher level than the level of CA 19-9 in pancreatic cystic lesions having no malignant potential.

15. A method for treating a pancreatic cystic lesion in a patient comprising:
obtaining a pancreatic cyst fluid sample from a patient having or suspected of having a pancreatic disease;
assaying the sample for a glycan level of MUC5AC in the sample;
comparing the glycan level in MUC5AC in the sample to a statistically validated threshold for MUC5AC, which statistically validated threshold for MUC5AC is based on a glycan level in MUC5AC in comparable control samples from benign pancreatic cysts in subjects; and
surgically removing the pancreatic cystic lesion from the patient if the glycan level in MUC5AC in the sample is different than the statistically validated threshold.

16. The method of claim 15, wherein the glycan level of MUC5AC is assayed with a lectin.

17. The method of claim 16, wherein the lectin is selected from the group consisting of Vicia villosa, Jacalin, wheat-germ agglutinin (WGA), and Erythrina cristagalli lectin (ECL).

18. The method of claim 15, wherein the sample is obtained by endoscopic ultrasound fine-needle aspiration.

19. The method of claim 15, further comprising:
assaying the sample for a level of CA 19-9;
comparing the glycan level of CA 19-9 in the sample to a statistically validated threshold for CA 19-9, which statistically validated threshold for CA 19-9 is based on a level of CA 19-9 in comparable control samples from benign pancreatic cysts in subjects; and
surgically removing the pancreatic cystic lesion from the patient if the glycan level in MUC5AC in the sample is different than the statistically validated threshold for MUC5AC, and if the level of CA 19-9 in the sample is different than the statistically validated threshold for CA 19-9.

20. A kit, comprising:
(a) an antibody microarray having an anti-CA 19-9 capture antibody and an anti-MUC5AC capture antibody bound thereto,
(b) a detection antibody to the CA-19-9 antigen,
(c) a detection antibody to a MUC5AC glycan, and
(d) one or more containers for such detection antibodies.
